(19) United States
(12) Patent Application Publication
(10) Pub. No.: US 2021/0059635 A1
(43) Pub. Date: Mar. 4, 2021

(54) TRANS-ESOPHAGEAL TONOMETRY

(71) Applicant: Mayo Foundation for Medical Education and Research, Rochester, MN (US)

(72) Inventors: Peter M. Pollak, Atlantic Beach, FL (US); Michael B. Wallace, Jacksonville, FL (US); Charles J. Bruce, Ponte Vedra Beach, FL (US)

(73) Assignee: Mayo Foundation for Medical Education and Research, Rochester, MN (US)

(21) Appl. No.: 16/644,273

(22) PCT Filed: Sep. 5, 2018
(86) PCT No.: PCT/US2018/049531

§ 371 Date: Mar. 4, 2020

Related U.S. Application Data
(60) Provisional application No. 62/554,281, filed on Sep. 5, 2017.

(51) Int. Cl. A61B 8/08; A61B 8/12; A61B 8/14; A61B 8/00; A61B 5/03; A61B 5/00

(52) U.S. Cl. CPC A61B 8/0883; A61B 8/12; A61B 5/0053; A61B 8/445; A61B 5/037; A61B 8/14

(57) ABSTRACT

Devices described herein can be used to directly measure left atrial pressure. For example, this document describes multiple embodiments of catheter-based, trans-esophageal tonometry devices that are used to directly measure left atrial pressure in a non-invasive manner.

### CROSS-REFERENCE TO RELATED APPLICATIONS

This application claims the benefit of U.S. Provisional Application Ser. No. 62/554,281, filed Sep. 5, 2017. The disclosure of the prior application is considered part of (and is incorporated by reference in) the disclosure of this application.

### BACKGROUND

### 1. Technical Field

This document relates to devices for measuring left atrial pressure and methods for their use. For example, this document relates to catheter-based, trans-esophageal tonometry devices that are used to measure left atrial pressure in a non-invasive manner.

### 2. Background Information

Direct measurements of left atrial pressure are typically invasive and relatively arduous to obtain. Due to such difficulties, estimates of the left atrial pressure are often used (as opposed to actual direct measurements). However, the estimates may lack the accuracy that clinicians and their patients often need for accurate diagnoses and effective treatments.

The law of Laplace uses factors such as wall tension, wall deflection, internal pressure and the radius of a thin wall vessel storing or transmitting fluid to provide a means to determine the internal pressure. In accordance with such principles, it has been shown that when a pressure transducer is used to flatten a curved wall by exerting an external pressure, the external pressure is essentially equal to the internal pressure. This technique is known as tonometry or applanation tonometry.

Applanation tonometry is essentially noninvasive, reproducible, and can provide an accurate representation of the aortic pressure waveform in many cases. Measurement of the aortic waveform by tonometry can provide clinicians with very useful information from which a patient's disease state can be determined. For example, in some cases heart problems and/or lung problems can be accurately diagnosed using aortic pressure waveforms obtained using applanation tonometry techniques.

### SUMMARY

This document describes devices for measuring left atrial pressure and methods for their use. For example, this document describes multiple embodiments of catheter-based, trans-esophageal tonometry devices that are used to directly measure left atrial pressure in a non-invasive manner.

In one aspect, this disclosure is directed to a trans-esophageal tonometry system. The trans-esophageal tonometry system includes: a trans-esophageal echocardiography (TEE) probe including an ultrasonic transceiver attached at a distal end portion of the TEE probe; a tonometry probe coupled to the distal end portion; and a force-measurement device operable to measure a force exerted by the tonometry probe.

Such a trans-esophageal tonometry system may optionally include one or more of the following features. The tonometry probe may be movably coupled to the distal end portion and thereby reconfigurable between a retracted low-profile delivery configuration and an extended operable configuration. The tonometry probe may be immovably coupled to the distal end portion. The tonometry probe may extend or may be extendable transversely from a longitudinal axis of the TEE probe. The tonometry probe may be positionable within the field of view of the ultrasonic transceiver. The tonometry probe may extend within a working channel of the TEE probe.

In another aspect, this disclosure is directed to a tonometry device. The tonometry device includes a housing releasably coupleable with a distal end portion of a trans-esophageal echocardiography (TEE) probe; a tonometry instrument coupled to the housing; and a force-measurement device operable to measure a force exerted by the tonometry instrument.

Such a tonometry device may optionally include one or more of the following features. The tonometry instrument may be movably coupled to the housing and thereby reconfigurable between a retracted low-profile delivery configuration and an extended operable configuration. The tonometry instrument may be pivotable in relation to the housing. The tonometry instrument may be immovably coupled to the housing. The tonometry device may also include a cable coupled to the housing and may also include one or more electrical wires that transmit a signal from the force-measurement device. The cable may include an electrical wire, an actuation cable, or a fluid tube by which the tonometry instrument is moveable relative to the housing between a retracted low-profile delivery configuration and an extended operable configuration.

In another aspect, this disclosure is directed to a method of measuring left atrial pressure of a patient. The method includes: (a) inserting a trans-esophageal echocardiography (TEE) probe within an esophagus of the patient to a position at which a distal end portion of the TEE probe is proximate to a left atrium of the patient; (b) obtaining ultrasonic images of a wall portion of the left atrium; (c) exerting pressure to the wall portion of the left atrium by a tonometry probe coupled to the distal end portion of the TEE probe; (d) measuring a deflection or flatness of the wall portion of the left atrium while the pressure is exerted to the wall portion of the left atrium; and (e) determining the left atrial pressure based on: (i) the measured deflection or flatness of the wall portion of the left atrium and (ii) the pressure exerted to the wall portion of the left atrium from the tonometry probe.

Such a method may optionally include one or more of the following features. The measuring may be synchronized with a respiratory cycle of the patient. The measuring may be synchronized with a cardiac cycle of the patient.

Particular embodiments of the subject matter described in this document can be implemented to realize one or more of the following advantages. In some embodiments, heart conditions such as diastolic heart failure and others can be accurately diagnosed using the devices and methods provided herein. In some embodiments, left atrial pressure can be directly measured in a non-invasive fashion using the devices and methods provided herein. For example, among other things, a puncture of the atrial septum can be avoided using the devices and methods described herein. Such non-invasive techniques can advantageously reduce recovery times, patient discomfort, patient risks, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of patient undergoing a trans-esophageal echocardiogram.

FIG. 2 is a perspective view of a distal end portion of an example trans-esophageal tonometry device in accordance with some embodiments provided herein.

FIG. 3 is a perspective view of an example tonometry device that can be coupled to an existing trans-esophageal echocardiogram probe.

FIG. 4 is a side view of the tonometry device of FIG. 3.

FIG. 5 is a flow chart of an example method for measuring left atrial pressure using a trans-esophageal tonometry device in accordance with some embodiments described herein.

Like reference numbers represent corresponding parts throughout.

### DETAILED DESCRIPTION

This document describes devices for measuring left atrial pressure and methods for their use. For example, this document describes multiple embodiments of catheter-based, trans-esophageal tonometry devices that are used to directly measure left atrial pressure in a non-invasive manner.

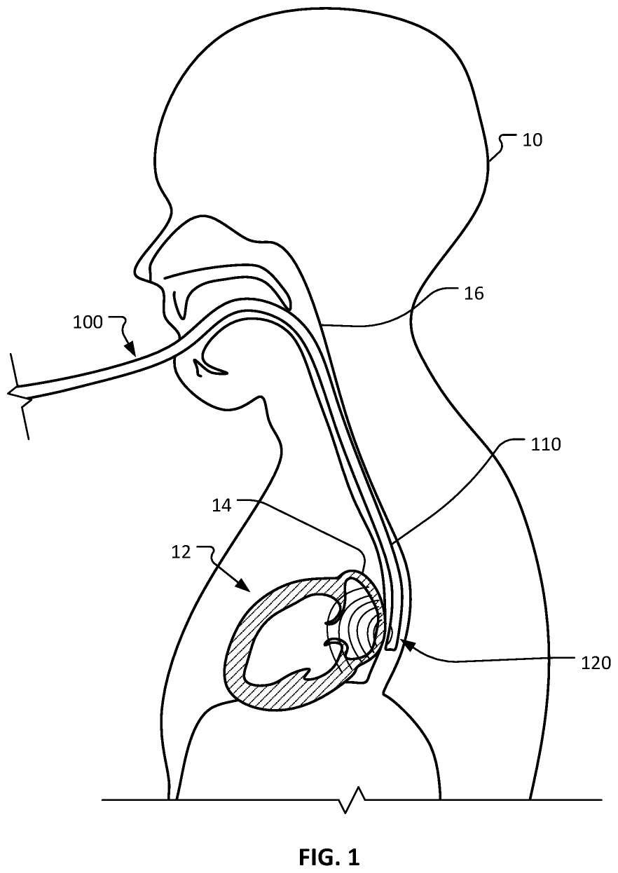

Referring to FIG. 1, a patient 10 is schematically depicted as undergoing a trans-esophageal echocardiography (TEE) procedure. The TEE procedure is performed using a TEE system 100. TEE system 100 includes a TEE probe 110 that includes a distal end portion 120. A proximal end of TEE probe 110 is operatively connected to an imaging system (not shown) that processes and displays ultrasonic images captured by distal end portion 120.

Patient 10 includes a heart 12 and an esophagus 16. Heart 12 includes a left atrium 14. As shown, esophagus 16 and left atrium 14 are generally in close anatomical proximity to each other (essentially in contact with each other, in some cases). Accordingly, esophagus 16 provides a beneficial access site from which to obtain ultrasound images of left atrium 14.

To perform the TEE procedure, TEE probe 110 is gradually inserted into esophagus 16. One or more portions of TEE probe 110 may be steerable (e.g., deflectable or articulable) by the imaging clinician to facilitate the insertion process. For example, in some embodiments distal end portion 120 may be steerable along one or more planes of deflection. The insertion continues until distal end portion 120 is adjacent to left atrium 14. In that orientation, ultrasonic images of left atrium 14 can be readily obtained by TEE system 100.

Such an orientation of distal end portion 120 relative to left atrium 14 (as depicted in FIG. 1) can also be exploited for performing direct measurements of left atrial pressure using tonometry. Heretofore, however, catheter-based tonometry devices for measuring left atrial pressure using a trans-esophageal approach have not been known.

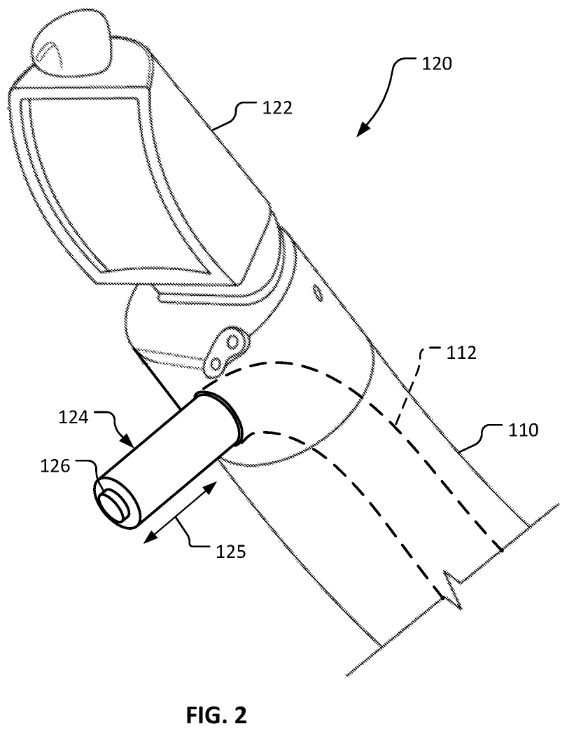

Referring also to FIG. 2, in some implementations a tonometry probe 124 can extend from distal end portion 120 of TEE system 100. For example, tonometry probe 124 can exit from an existing accessory channel 112 of TEE probe 110 and directionally extend from distal end portion 120 such that an ultrasound transceiver 122 of TEE system 100 can capture ultrasound images of tissues that tonometry probe 124 makes contact with.

In some embodiments, tonometry probe 124 is an elongate instrument that can be extended through a working channel 112 of TEE probe 110. In some embodiments, tonometry probe 124 is permanently coupled to distal end portion 120 of TEE probe 110. In some embodiments, tonometry probe 124 can be selectively extended generally transversely (radially) from a longitudinal axis of TEE probe 110, as indicated by arrow 125.

In use, tonometry probe 124 can be manipulated to exert force to a wall portion of left atrium 14 (with an intervening portion of esophagus wall there between). Such a force from tonometry probe 124 will depress and flatten to a certain extent the otherwise curved wall portion of left atrium 14. The extent to which the left atrial wall is depressed and/or flattened resulting from force exerted by tonometry probe 124 can be measured using the imaging capabilities of TEE system 100. Additionally, the force exerted by a depressible member 126 of tonometry probe 124 to create the depression and/or flatness of the left atrial wall can be measured or otherwise known. In that manner, the law of Laplace can be used to indirectly measure the pressure within left atrium 14.

In some embodiments, tonometry probe 124 is movably coupled with and selectively deployable relative to TEE probe 110. That is, as depicted by arrow 125, in some embodiments tonometry probe 124 is movably coupled to TEE probe 110 such that tonometry probe 124 can be extended transversely from TEE probe 110, and can be transversely retracted to within or essentially within the outer diameter of TEE probe 110. In some such embodiments, the orientation of tonometry probe 124 relative to TEE probe 110 can be controlled by a clinician operator of TEE system 100. Tonometry probe 124 can be actuated radially inward or outward using various mechanisms such as, but not limited to, a mechanical linkage, a fluid-actuated cylinder (e.g., an air cylinder), an inflatable bladder, an electrical solenoid, electromagnetism devices, a spring mechanism, a screw mechanism, and the like.

Pressure can be applied by depressible member 126 of tonometry probe 124 to tissue surfaces (e.g., to a wall portion of the left atrium 14). In some embodiments, the pressure is created by the selective deployment of tonometry probe 124. In some embodiments, depressible member 126 is spring-loaded such that a particular, known pressure can be applied by depressible member 126. In some embodiments, the capability to steer/deflect distal end portion 120 is used to exert pressure by tonometry probe 124 to tissue surfaces.

TEE system 100 includes functionality for ascertaining the force exerted by tonometry probe 124. For example, in some embodiments one or more load cells or strain gauge devices (e.g., microscale strain gauges, piezoresistors, capacitive strain gauges, and the like) are coupled to tonometry probe 124 (e.g., coupled to depressible member 126) to measure the force exerted by tonometry probe 124. In some embodiments, fluid pressure (e.g., liquid or gas) or a spring is used to measure the force exerted by tonometry probe 124 (e.g., by depressible member 126). Other suitable types of devices and techniques for measuring force can also be used.

Tonometry probe 124 is oriented so that the image captured by ultrasound transceiver 122 (the field of view of ultrasound transceiver 122) includes the tissue wall to which force from tonometry probe 124 is applied. Accordingly, the resulting depression/flatness of the tissue wall (e.g., wall portion of left atrium 14) can be captured by ultrasound transceiver 122 (and TEE system 100 as a whole). In result, TEE system 100 can be used to measure the extent of the depression/flatness of the tissue wall caused by the force from tonometry probe 124.

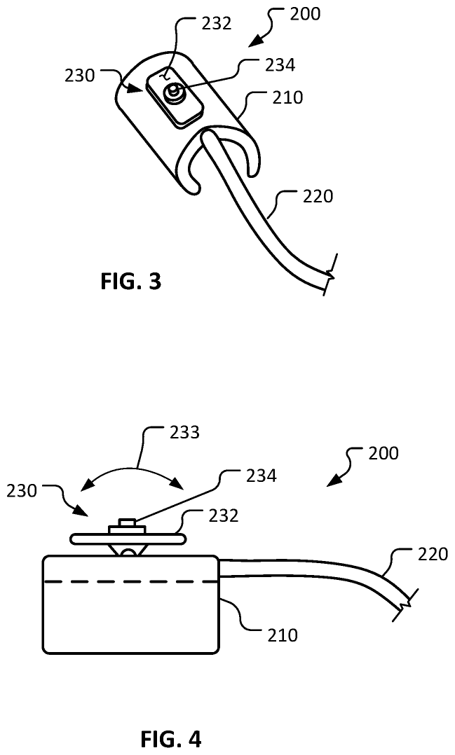

Referring also to FIGS. 3 and 4, in some embodiments an example tonometry device 200 can be releasably coupleable to the outer diameter of a probe of a standard TEE system. Tonometry device 200 includes a housing 210, a control cable 220, and a tonometry instrument 230.

As shown, tonometry instrument 230 extends from housing 210 so that tonometry instrument 230 can be pressed against a wall portion of left atrium 14 (with an intervening portion of esophagus wall there between). Such a force from tonometry instrument 230 will depress and flatten to a certain extent the otherwise curved wall portion of left atrium 14. The extent to which the left atrial wall is depressed and/or flattened resulting from force exerted by tonometry instrument 230 can be measured using the imaging capabilities of the TEE system to which tonometry device 200 is coupled. Additionally, the force exerted by a depressible member 234 of tonometry instrument 230 to create the depression and/or flatness of the left atrial wall can be measured or otherwise known. In some embodiments, a spring is included to bias depressible member 234 towards the extended configuration as shown. In that manner, the law of Laplace can be used to indirectly measure the pressure within left atrium 14.

In some embodiments, tonometry instrument 230 can be selectively deployed between a low-profile retracted configuration (e.g., as would be used during insertion and retraction of tonometry device 200) and a radially extended configuration (e.g., as would be used during actual tonometry measurements). The movements of tonometry instrument 230 relative to housing 210 can be actuated using various mechanisms such as, but not limited to, a mechanical linkage, a Bowden cable, a fluid-actuated cylinder (e.g., an air cylinder), an inflatable bladder, an electrical solenoid, electromagnetism devices, a spring mechanism, a screw mechanism, and the like, and combinations thereof.

In some embodiments, tonometry instrument 230 is coupled to housing 210 in a fixed relationship (essentially not radially movable). In the depicted embodiment, tonometry instrument 230 is permanently or fixedly radially-extended from housing 210 in an operable configuration (e.g., as would be used during actual tonometry measurements).

Housing 210 is configured to facilitate tonometry device 200 being coupled to and decoupled from a probe of a standard TEE system. For example, in the depicted embodiment housing 210 has a c-shaped cross-sectional profile by which housing 210 can snap onto and clamp onto a TEE probe. In some embodiments, housing 210 has an annular ring cross-sectional profile. In some embodiments, other mechanical structures are used to couple tonometry device 200 with a probe of a standard TEE system. For example, housing 210 can be a two-piece housing assembled around a probe to clamp thereon, housing 210 can be a hollow cylindrical shape that engages with a probe using a frictional fit, housing 210 can include a rotatable collet-like lock collar, and the like.

Control cable 220 extends from housing 210. In some implementations, control cable 220 is coupled to the probe of the standard TEE system. Control cable 220 can include electrical wires and/or fluid tubes. For example, the electrical wire(s) used to transmit a signal indicative of force applied by tonometry instrument 230 (e.g., from a load cell coupled to depressible member 234) can run through cable 220. In addition, wires, cables, or fluid tubes used to actuate movements of tonometry instrument 230 between the retracted and extended configurations can be run through cable 220.

In some embodiments, tonometry instrument 230 is movably coupled to housing 210 so that tonometry instrument 230 will naturally tend to be presented to the abutting tissue surface (e.g., esophageal wall) generally orthogonally. For example, in the depicted embodiment a platform 232 of tonometry instrument 230 is pivotably coupled to housing 210. Platform 232 is pivotable in relation to housing 210 as indicated by double-arrow 233.

Tonometry device 200 includes functionality for ascertaining the force exerted by tonometry instrument 230. For example, in some embodiments one or more load cells or strain gauge devices (e.g., microscale strain gauges, piezoresistors, capacitive strain gauges, and the like) are coupled to tonometry instrument 230 (e.g., to depressible member 234) to measure the force exerted by tonometry instrument 230. In some embodiments, a spring or fluid pressure (e.g., liquid or gas) is used to measure the force exerted by tonometry instrument 230. Other suitable types of devices and techniques for measuring force can also be used.

In use, tonometry device 200 is releasably coupled to a TEE probe in a relative position so that the image captured by the ultrasound transceiver of the TEE probe (the field of view of ultrasound transceiver) includes the tissue wall to which force from tonometry instrument 230 is applied. Accordingly, the resulting depression/flatness of the tissue wall (e.g., wall portion of left atrium 14) can be captured by the ultrasound transceiver (and TEE system as a whole). In result, the TEE system to which tonometry device 200 is attached can be used to measure the extent of the depression/flatness of the tissue wall caused by the force from tonometry instrument 230. The measured depression/flatness of the tissue wall, along with the measured/known force applied by tonometry instrument 230 (e.g., by depressible member 234) can be used to determine the internal pressure of a chamber (e.g., the left atrium).

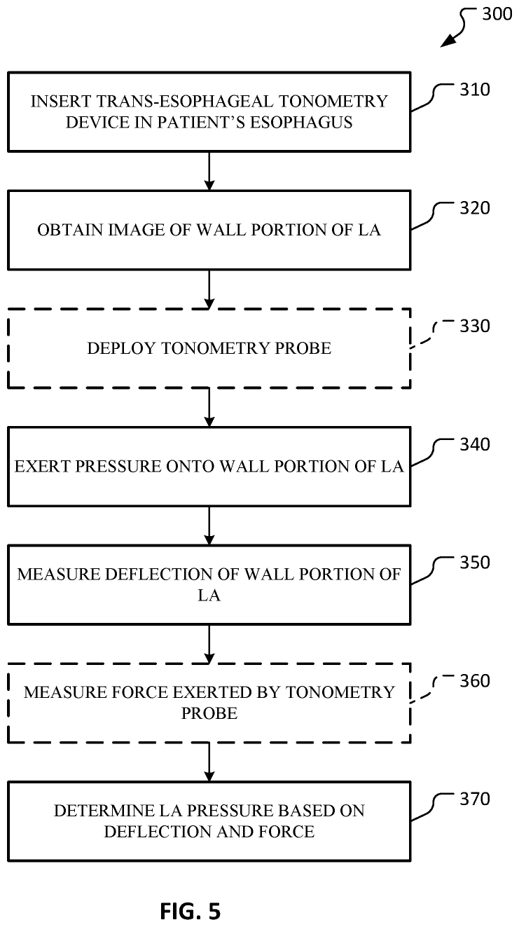

FIG. 5 provides a flow chart of a method 300 for using catheter-based, trans-esophageal tonometry devices to directly measure left atrial pressure in a non-invasive manner.

Method 300 begins at step 310 which comprises insertion of a trans-esophageal tonometry device in patient's esophagus (e.g., to arrive at the orientation shown in FIG. 1). The trans-esophageal tonometry device can be a TEE system with a permanently attached tonometry probe located at a distal end portion 120 (e.g., FIG. 2) or a conventional TEE system to which is releasably attached a tonometry device 200 (FIGS. 3 and 4).

At step 320, an image of a wall portion of the left atrium (LA) is captured using the TEE system.

At optional step 330, a tonometry probe is deployed. For example, the tonometry probe can be transversely deployed (e.g., as depicted by arrow 125 of FIG. 2 or pivoted as depicted in FIGS. 3 and 4). In some implementations, the tonometry probe is affixed in a stationary relationship to other portions of the tonometry device (therefore, this step is optional).

At step 340, pressure from the tonometry probe is exerted onto the wall portion of the left atrium. The pressure is transferred to the wall portion of the left atrium through the wall of the esophagus. Enough pressure is exerted onto the wall portion of the left atrium to achieve a desired extent of depression/flattening of the wall portion of the left atrium.

At step 350, the extent of depression/flattening of the wall portion of the left atrium resulting from the pressure exerted by the tonometry probe is measured. The measurement can be performed using the TEE system.

At optional step 360, the force exerted by the tonometry probe to cause the measured depression/flattening of the wall portion of the left atrium is measured. In some implementations, the tonometry system is designed to exert a fixed amount of force, so the force exerted by the tonometry probe is known (therefore, this step is optional).

At step 370, the left atrial pressure is determined based on the measured deflection/flattening (from step 350) and the force exerted by the tonometry probe (from step 360, or from a known value). This step can be performed using an algorithm run by a computerized device. The computerized device can also display the determined left atrial pressure value.

In some embodiments, the measurement at step 350 is synchronized with the patient's respiratory cycle. In particular embodiments, the measurement at step 350 is synchronized with the patient's cardiac cycle. In some embodiments, one or more transient pressures from tonometry probe is/are applied (as opposed to a prolonged application of pressure). In some such cases, multiple measurements of deflection and force may be obtained and combined (e.g., averaged) to attain a representative value of the left arterial pressure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A trans-esophageal tonometry system, comprising:
a trans-esophageal echocardiography (TEE) probe including an ultrasonic transceiver attached at a distal end portion of the TEE probe;
a tonometry probe coupled to the distal end portion; and
a force-measurement device operable to measure a force exerted by the tonometry probe.

2. The system of claim 1, wherein the tonometry probe is movably coupled to the distal end portion and thereby reconfigurable between a retracted low-profile delivery configuration and an extended operable configuration.

3. The system of claim 1, wherein the tonometry probe is immovably coupled to the distal end portion.

4. The system of claim 1, wherein the tonometry probe extends or is extendable transversely from a longitudinal axis of the TEE probe.

5. The system of claim 4, wherein the tonometry probe is positionable within the field of view of the ultrasonic transceiver.

6. The system of claim 1, wherein the tonometry probe extends within a working channel of the TEE probe.

7. A tonometry device, comprising:
a housing releasably coupleable with a distal end portion of a trans-esophageal echocardiography (TEE) probe;
a tonometry instrument coupled to the housing; and
a force-measurement device operable to measure a force exerted by the tonometry instrument.

8. The device of claim 7, wherein the tonometry instrument is movably coupled to the housing and thereby reconfigurable between a retracted low-profile delivery configuration and an extended operable configuration.

9. The device of claim 7, wherein the tonometry instrument is pivotable in relation to the housing.

10. The device of claim 7, wherein the tonometry instrument is immovably coupled to the housing.

11. The device of claim 7, further comprising a cable coupled to the housing and including one or more electrical wires that transmit a signal from the force-measurement device.

12. The device of claim 11, wherein the cable includes an electrical wire, an actuation cable, or a fluid tube by which the tonometry instrument is moveable relative to the housing between a retracted low-profile delivery configuration and an extended operable configuration.

13. A method of measuring left atrial pressure of a patient, the method comprising:
inserting a trans-esophageal echocardiography (TEE) probe within an esophagus of the patient to a position at which a distal end portion of the TEE probe is proximate to a left atrium of the patient;
obtaining ultrasonic images of a wall portion of the left atrium;
exerting pressure to the wall portion of the left atrium by a tonometry probe coupled to the distal end portion of the TEE probe;
measuring a deflection or flatness of the wall portion of the left atrium while the pressure is exerted to the wall portion of the left atrium; and
determining the left atrial pressure based on: (i) the measured deflection or flatness of the wall portion of the left atrium and (ii) the pressure exerted to the wall portion of the left atrium from the tonometry probe.

14. The method of claim 13, wherein the measuring is synchronized with a respiratory cycle of the patient.

15. The method of claim 13, wherein the measuring is synchronized with a cardiac cycle of the patient.
